(19) United States
(12) Patent Application Publication
(10) Pub. No.: US 2025/0336487 A1
(43) Pub. Date: Oct. 30, 2025

(54) reconstruction method and system of aerosol chemical components based on CNN-BiLSTM-BO

(71) Applicant: Institute of Atmospheric Physics, Chinese Academy of Sciences, Beijing (CN)

(72) Inventors: Ting YANG, Beijing (CN); Hongyi LI, Beijing (CN); Zifa WANG, Beijing (CN); Yining Tan, Beijing (CN)

(21) Appl. No.: 18/777,007

(22) Filed: Jul. 18, 2024

(30) Foreign Application Priority Data
Apr. 30, 2024 (CN) 2024105442971

(51) Int. Cl. G16C 20/70; G06N 3/0464

(52) U.S. Cl. CPC G16C 20/70; G06N 3/0464

(57) ABSTRACT

A method and a system for reconstructing aerosol chemical components based on CNN-BiLSTM-BO, including collecting multi-source environmental observation data through observation equipment, preprocessing and extracting key characteristic variables. The pre-treated multi-source environmental observation data are input into the CNN-BILSTM model for feature analysis, and the CNN-BiLSTM hyperparameters are adjusted by Bayesian optimization algorithm to generate a reconstructed model of aerosol chemical components. After verifying the performance and stability of the reconstructed model, the predicted results of the chemical components of the aerosol are output. On the basis of not relying on traditional chemical analysis technology, the invention can accurately reconstruct various aerosol chemical components, greatly reduce the cost and time of chemical analysis, effectively solve the problems of variable inconsistency, data missing, and spatio-temporal mismatch in multi-source observation data, and automatically adjust hyperparameters through Bayesian optimization algorithm to ensure that the output prediction results are more accurate.

### FIELD OF THE INVENTION

This invention relates to the field of aerosol detection, in particular to a method and system for reconstructing aerosol chemical components based on CNN-BILSTM-BO.

### BACKGROUND OF THE INVENTION

From 2013 to 2017, China implemented the Air Pollution Prevention and Control action Plan, referred to as the “Air Ten”. Since then, from 2018 to 2020, the “Blue Sky Defense” campaign has continued to promote emission reduction and pollution prevention. Although aerosol pollution decreased significantly, after 2018, the decline trend of aerosol concentration slowed down, and in 2023, the aerosol concentration in most provincial capitals increased year-on-year, indicating that pollution control needs to pay attention to refined prevention and control. Aerosols contain sulfate (SO42—), nitrate (NO3—), ammonium salt (NH4+), organic matter (OM), and elemental carbon (EC) or black carbon (BC), which have different sources, formation mechanisms, and impacts on climate and health. For example, BC and SO42− have a significant effect on dementia, while NO3− and OC have a smaller effect. Therefore, accurate representation of the characterization of aerosol chemical components is important for developing emission reduction strategies and assessing health and climate effects.

In the existing technology, the detection technology of aerosol components mainly relies on chemical analysis and numerical simulation. Chemical analyses such as ion chromatography and thermal/optical reflection are accurate but costly and are limited by low spatio-temporal resolution and instrument accuracy. In addition, atmospheric chemical transport models are susceptible to uncertainties such as emission inventories, meteorological fields, physico-chemical mechanisms, and initial boundary conditions. Although numerical simulation can simulate the formation and propagation of aerosols, it is easily affected by uncertain factors such as emission inventory and meteorological conditions. In contrast, machine learning techniques based on probability theory and mathematical statistics offer a more efficient approach that is not subject to these limitations, and machine learning is divided into supervised learning, unsupervised learning, and reinforcement learning. Among them, supervised learning is suitable for establishing relationships between the input and the output, but linear algorithms such as ARIMA and MLR and GWR algorithms cannot capture complex nonlinear relationships, and their accuracy and generalization ability are limited. Traditional nonlinear algorithms such as SVM and random forest have improved this, but the accuracy, generalization and interpretability of deep learning techniques such as CNN and LSTM in characterizing aerosol chemical components need to be improved.

### BRIEF SUMMARY OF THE INVENTION

Aiming at the above technical problems, the invention provides a method and system for reconstructing aerosol chemical components based on CNN-BILSTM-BO, and on the basis of independent traditional chemical analysis technology, accurately reconstruct a variety of aerosol chemical components (ammonium salt NH4+, sulfate SO42—, nitrate NO3—, organic matter OM and elemental matter EM), and solve the problems of high observation cost of chemical components, data missing, and inconsistent variable types of multi-source observation data. The purpose of this invention can be realized through the following technical solutions:

This invention provides a reconstruction method of aerosol chemical components based on CNN-BILSTM-BO, comprising the following steps:
Step S1. Collect multi-source environmental observation data through observation equipment, and conduct pre-processing and extract key feature variables;
Step S2. Input the pre-processed multi-source environment observation data into the CNN-BiLSTM model for feature analysis, wherein, convolutional neural network extracts spatial features and uses bidirectional long short-term memory neural network to process time series information, and adjusts hyperparameters of the CNN-BiLSTM through Bayesian optimization algorithm. And then a reconstructed model of aerosol chemical components is generated.
Step S3. After verifying the performance and stability of the reconstructed model, outputs the predicted results of the chemical components of the aerosol.

Further, in step S2, the convolutional neural network extracts spatial features, including folding the multivariable time series of the multi-source environment observation data into a multivariable array to complete independent convolution operations at each time step. Each time step of the multi-source environment observation data is independently convolved along the horizontal and vertical directions, local features are extracted by convolution kernel and normalized and nonlinear processing is performed by batch normalization and correction of linear elements. The time series structure of the multi-source environment observation data is reconstructed using the sequence expansion layer, and the spatial dimension is folded into the variable dimension by the flattening layer.

Further, in step S2, the bidirectional long short term memory neural network is used to process the time series information, including, the bidirectional long short term memory layer inputs the time features in the data from the convolutional neural network through forward and backward ways, and simultaneously captures the past and future information. Randomly remove a certain percentage of neurons by dropping layers. The output result of the bidirectional long short term memory layer is dimensionally transformed to match the target output size using the fully connected layer, and finally the regression output layer outputs the regression estimate of the aerosol chemical component.

Further, in step S2, the hyperparameters are adjusted by the Bayesian optimization algorithm, including,

The objective function of the CNN-BILSTM model is constructed, and a set of initial hyperparameters are randomly selected in the decision space as the initial decision vector, the value of the objective function is calculated and the prior probability distribution is obtained, and the maximum number of iterations and calculation time are controlled by the control parameters.

The likelihood distribution of the Gaussian process regression model of the objective function is updated by the actual observed values, and the posterior probability distribution is derived to reflect the update of the objective function.

Based on the posterior probability distribution, the acquisition function value is maximized to determine the next optimal sampling point, and the corresponding prior probability distribution is continued to be updated, iterating continuously until the combination of hyperparameters corresponding to the minimum value of the objective function is determined, and the hyperparameter combination is the corresponding optimal solution.

Further, in step S211, construct the objective function of the CNN-BILSTM model, randomly select a set of initial hyperparameters in the decision space as the initial decision vector, calculate the value of the objective function and obtain the prior probability distribution, including,

$$\begin{matrix}p ( f ( x \vert input ) \vert Y ) = p ( Y \vert f ( x \vert input ) ) p ( f ( x \vert input ) ) / p ( Y ) ; \\ {x}^{*} = arg min f ( x \vert input ) , x ∈ X ⊆ {R}^{d} ;\end{matrix}$$

Where, x is the decision vector composed of d hyperparameters, X is the decision space, input is the input data, x* is the optimal hyperparameter combination vector. f(x|input) is the objective function, Y is the actual observation value, p(Y|f(x|input)) is the likelihood distribution, p(f(x|input)) is the prior probability distribution, that is, the estimate of f(x|input), p(Y) is the edge likelihood distribution, p(f(x|input)|Y) is the posterior probability distribution, that is, the confidence coefficient of f(x|input).

Further, in step S211, the likelihood distribution of the Gaussian process regression model of the objective function is updated by the actual observed values, and a posterior probability distribution is derived from it to reflect the update of the objective function, including,

$$\begin{matrix}f ( x \vert input ) \sim GP ( mean ( x ) , k ( x , {x}^{′} ; θ ) ) ; \\ k ( x , {x}^{′} ; θ ) = {θ}_{1} ( 1 + \frac{\sqrt{5} \vert x - {x}^{′} \vert}{{θ}_{2}} + \frac{5 {( x - {x}^{′} )}^{2}}{3 {θ}_{2}} ) exp ( - \frac{\sqrt{5} \vert x - {x}^{′} \vert}{{θ}_{2}} ) ; \\ {θ}_{1} = {σ}_{f}^{2} , {σ}_{f} > 0 ; \\ {θ}_{2} = {σ}_{1} , {σ}_{1} > 0 ;\end{matrix}$$

Where GP(mean(x),k(x,x′;θ) is the Gaussian process regression model, consisting of the mean function (mean(x)) and the covariance kernel function (k(x,x′;θ)) composition. The k(x,x′;θ) is the kernel function of the Matern 5/2 covariance, where x and x′ are expressed as any two coordinate points, θ is the kernel parameter vector, σ_{f} is the signal standard deviation, and σₗ is the feature length scale.

Further, based on the posterior probability distribution, the acquisition function value is maximized to determine the next optimal sampling point, and the corresponding prior probability distribution is continuously updated, iterating until the combination of hyperparameters corresponding to the minimum value of the objective function is determined, and the combination of hyperparameters is the corresponding optimal solution, including,

$$\begin{matrix}EI ( x , P ) = {E}_{P} [ max ( 0 , {μ}_{P} ( {x}_{best} ) - f ( x \vert input ) ) ] ; \\ EIpS ( x ) = {EI}_{P} ( x ) / {μ}_{s} ( x ) ; \\ {σ}_{P}^{2} ( x ) = {σ}_{F}^{2} ( x ) + {σ}^{2} , {σ}_{F} ( x ) < {t}_{σ} σ ;\end{matrix}$$

Where EI(x,P) is the collection function, x is the same as x in the objective function(f(x|input)), P is the objective function (p(f(x|input))), μ_{P}(x_{best}) is the least posterior mean, and x_{best} is the currently known optimal solution. ElpS(x) represents the expected improvement function per second, and μₛ(x) is the posterior mean of the time Gaussian process model.

$${σ}_{p}^{2} ( x )$$

represents the addition function, σ_{F}(x) is the standard F deviation of the f(x|input), σ is the posterior standard deviation of the additional noise, and t_{σ} is the value of the extraction proportion in the acquisition function.

Based on the same invention idea, this invention also provides a reconstruction system of aerosol chemical components based on CNN-BILSTM-BO, which adopts the reconstruction method of aerosol chemical components as described above, including,

The data preprocessing module and the initialization system, which can collect multi-source environment observation data, preprocess and extract key feature variables, and define the decision space of control parameters and hyperparameter optimization.

In the feature analysis and evaluation module, the pre-processed multi-source environment observation data is input into the CNN-BILSTM model for feature analysis. In this module, the convolutional neural network extracts spatial features, and uses the bidirectional long short-term memory neural network to process time series information, and evaluates the performance of the model.

The model training optimization module iteratively adjusts the hyperparameters of the CNN-BILSTM model by Bayesian optimization algorithm, trains the model, generates the optimal reconstructed model and outputs the prediction results of aerosol chemical components.

Further, in the feature analysis and evaluation module, the multi-variable time series of the multi-source environment observation data is converted into a multi-variable array through data folding, so that each time step is carried out independent convolution operation, the convolution layer extracts local features along the horizontal and vertical directions, and ensures the stability of the output through batch normalization processing. The modified linear unit performs nonlinear processing on the data after batch normalization, the average pooling layer divides the data into multiple regions, the sequence expansion layer restores the time series structure, the flattening layer converts the spatial dimension into the variable dimension, and the BiLSTM layer processes the time series data in a forward and backward way to capture the past and future information. Finally, the discard layer randomly removes a certain proportion of neurons, and the fully connected layer converts the output results into dimensions. Finally, the regression output layer outputs the predicted results of aerosol chemical components.

Further, in the model training optimization module, a set of initial hyperparameters are randomly selected as the decision vector, and the initial values of the hyperparameters are calculated through the prior probability distribution of the objective function, and the likelihood distribution of the Gaussian process regression model is updated using the actual observed values. The posterior probability distribution of the objective function is derived, and based on the posterior probability distribution. The value of the acquisition function is maximized, the optimal sampling point is determined, and the prior probability distribution of the objective function is updated continuously, and the optimal hyperparameter combination corresponding to the minimum value of the objective function is finally obtained into the optimal solution.

Compared with the prior technology, this invention has at least one of the following technical effects:

The invention can accurately reconstruct a variety of aerosol chemical components (such as ammonium salt, sulfate, nitrate, organic matter and elemental matter) without relying on traditional chemical analysis techniques, and greatly reduces the cost and time of chemical analysis. At the same time, the deep neural network technology is used to effectively solve the problems of variable inconsistency, data missing, and space-time mismatch in the multi-source observation data, so as to ensure the stable operation of the model. In addition, the performance of the model is optimized through the automatic adjustment of hyperparameters by Bayesian optimization algorithms, ensuring that the output predictions are more accurate. By integrating feature analysis function, the system can extract and screen features of data, and further improve the flexibility and applicability of the model. The design of modular processing provides the possibility for the secondary development and perfection of the system, and enhances the flexibility and expansibility of the whole model.

### BRIEF DESCRIPTION OF THE DRAWINGS

In order to more clearly state the technical scheme of the embodiment of the invention, the following is a simple introduction of the drawings required in the description of the embodiment:

FIG. 1 is a step flow chart of the reconstruction method of aerosol chemical components based on CNN-BILSTM-BO of the invention.

FIG. 2 is a structural flow chart of the aerosol chemical component reconstruction system based on CNN-BILSTM-BO of the invention.

### DETAILED DESCRIPTION

In order to make the purpose, technical scheme and advantages of this invention more clear, the invention is further explained in detail in combination with the attached drawings and specific embodiments. It should be understood that the specific embodiments described herein are intended only to explain the invention and are not intended to qualify it.

### Embodiment 1

Aiming at the above technical problems, the invention proposes a reconstruction method of aerosol chemical components based on CNN-BILSTM-BO, which is used for the normalization and fusion of multi-source data, establishes the complex nonlinear mapping relationship between multi-source observed variables and the key chemical components of PM2.5, and maps the multi-source observed variables to the key chemical components of PM2.5, so that it can obtain the chemical component information of PM2.5 quickly and accurately. Deep neural networks (DNNs) have obvious advantages in dealing with nonlinear problems among multiple variables. The development of the reconstruction model of key chemical components of PM2.5 based on deep neural networks can effectively and cooperatively deal with the problems of variable inconsistency, spatio-temporal mismatch, and data missing of multi-source data. In the present invention, in order to fully and deeply mine the spatiotemporal features and inter-variable features of multi-source data, the reconstructed model is coupled by a convolutional neural network (CNN) for capturing spatial features and inter-variable features and a bidirectional long short-term memory neural network (BiLSTM) for processing time series information transmission (CNN-BILSTM). Coupled with the Bayesian optimization algorithm, the hyperparameters of the reconstructed model are automatically optimized to ensure the optimal performance of the model. The purpose of the invention can be realized through the following technical solutions:

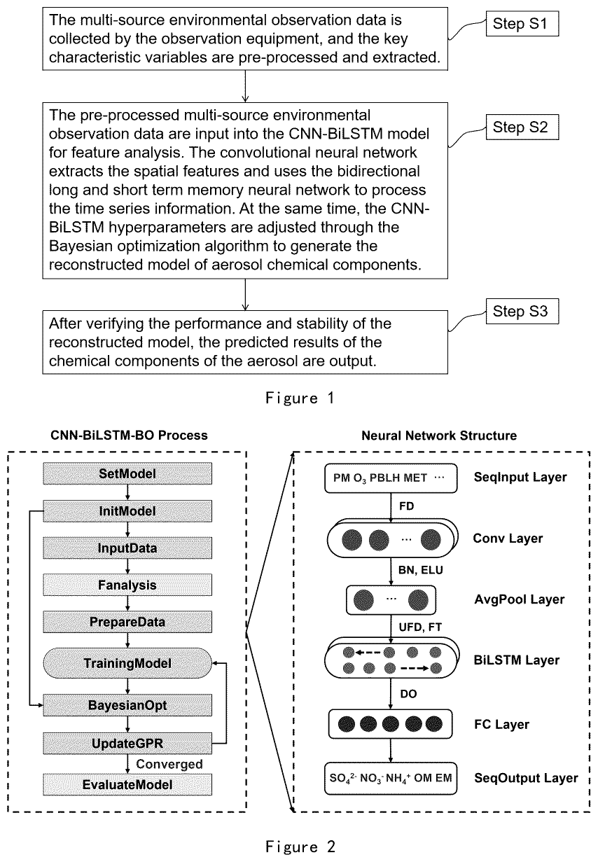

As shown in FIG. 1, the invention provides a reconstruction method of aerosol chemical components based on CNN-BILSTM-BO, including the following steps:
Step S1. Collect multi-source environmental observation data through observation equipment, conduct pre-processing and extract key feature variables.

In order to collect multi-source environmental observation data, we obtain various types of environmental information through a variety of observation equipment. These include weather monitors, particulate matter monitors, chemical composition analyzers and other observation tools. Each device can measure specific variables such as temperature, humidity, wind speed, wind direction, particulate concentration, chemical composition, and more. These data are the basis of constructing aerosol chemical component reconstruction model. After collecting the data, we first preprocess it. This includes data cleaning, denoising and standardising. The cleaning procedure is used to remove incorrect or abnormal data points to ensure data accuracy and consistency. After data preprocessing, key feature variables are extracted. This process involves identifying the variables most relevant to the reconstruction of aerosol chemical components from multi-source data, such as particulate matter concentration, meteorological elements, and atmospheric chemical components.
Step S2. Input the pre-processed multi-source environmental observation data into the CNN-BILSTM model for feature analysis. In this process, convolutional neural network extracts spatial features, uses bidirectional long short-term memory neural network to process time series information, and adjusts CNN-BiLSTM hyperparameters through Bayesian optimization algorithm, finally a reconstructed model of aerosol chemical components is generated. The specific process is as follows: the multi-variable time series of the multi-source environment observation data is folded into a multi-variable array, and the independent convolution operation is carried out at each time step. The convolution layer performs independent convolution calculation for each time step of the input multi-source environment observation data along the horizontal and vertical directions to extract local features. The convolution calculation includes multiplying the convolution kernel with the multi-source environment observation data and adding a bias term, and performs batch normalization processing for the convolutional layer output. The formula is:

$${c}_{t} = max ( 0 , f ( w \times {x}_{t} + {b}_{t} ) )$$

Where, cₜ and xₜ are the output and input of the convolution layer respectively, f(w×xₜ+bₜ) is the value after the convolution calculation, w and bₜ are the weight matrix and bias term of the convolution kernel respectively. The modified linear unit processes the data after batch normalization by introducing nonlinear parameters to the negative value of input and preserving the original value of the positive value. The pooling layer divides the two-dimensional input of the modified linear unit into multiple regions and averages the pooling. The time series structure of multi-source environmental observation data is recovered by the sequence expansion layer, and the spatial dimension is folded into the variable dimension by the flattening layer.

In the present invention, CNN is used to process time series data of different variables. The CNN is mainly composed of a convolutional layer and a pooled layer, in which the convolutional layer performs convolutional computation operations and is nonlinear processed by rectified linear units (ReLU, a kind of excitation function) to enhance the interpretability of the neural network model and reduce model overfitting. The pooling layer is responsible for nonlinear downsampling to preserve the main features and to reduce the parameters through maximum or average pooling of subdomains, thus reducing overfitting of the model.

The bidirectional long short term memory layer convolves the time features in the input data of the neural network in a forward and backward way to capture the past and future information at the same time. Randomly remove a certain percentage of neurons by dropping layers. The full connection layer is used to transform the output of the bidirectional long short term memory layer to match the target output size, and finally the output layer is used to output the regression estimate of aerosol chemical components.

In step S2, hyperparameters are also adjusted by Bayesian optimization algorithm. As the configuration parameters of the machine learning model, hyperparameters determine the neural network structure, initialization process, training process, testing process and convergence performance of the machine learning model, and directly affect the simulation and prediction results of the model. Common hyperparameter optimization methods include manual empirical parameter tuning, grid search and random search, etc. These algorithms are usually time-consuming, require a lot of computing resources, and may fall into local optimal solutions when dealing with non-convex problems. Compared with the above algorithms, Bayesian optimization provides a more efficient optimization framework, which is optimized by constructing a posterior distribution of the objective function and searching the global optimal solution with a smaller number of iterations, thus reducing the computational cost of evaluating the loss function and effectively improving the accuracy of the machine learning model. The basic principle of Bayesian optimization is to take the hyperparameters that need to be optimized in the machine learning model as decision variables to participate in the construction of the objective function, and transform the optimization problem into a function problem corresponding to the optimal solution when solving the minimum value of the objective function ƒ(x|input). Bayesian optimization consists of a probabilistic proxy model and a collection function, wherein the probabilistic proxy model is used to estimate the probability distribution of ƒ(x|input), thus approximately replacing the complex objective function and reducing the computational cost.

The Gaussian process regression model presented in this paper is a non-parametric model, which does not rely on specific distribution assumptions for the data, that is, there is no fixed function form, but is iteratively updated during the process of learning the data, which means that the Gaussian process regression model has excellent performance in dealing with complex nonlinear problems. The prediction of Gaussian process regression model follows normal distribution, which is composed of mean function and covariance kernel function k(x,x′;θ). The acquisition function is responsible for determining the most appropriate sampling point (potential optimal decision variable) in the iterative optimization process, which is used to update the probabilistic proxy model and improve the construction of the objective function ƒ(x|input). The basic principle of the collection function is to determine the next most promising sampling point by maximizing the value of the function for updating the probabilistic proxy model, which should meet the requirements of the lowest computing cost, the most significant improvement in confidence and the best fitting effect. The invention selects the “Expected-Improvement-Per-Second-Plus” (Elps+) function as the acquisition function. The collection function consists of three sub-functions, namely “expected improvement”, “Per Second”, and “add”. Among them “expected improvement” function is the core, the degree of improvement is expressed by difference between the known n−1 sample points of the optimal solution μ_{P}(x_{best}) and sampling of function value ƒ(x|input), and it is improved by taking maximization x to determine the next sampling points. The “per second” function is used to measure the computing time of the objective function in the collection function. While taking the improvement degree as the evaluation index, the computing time of the objective function is coordinated to avoid the high time cost and computing resources caused by the calculation of the objective function in the actual optimization process. The “plus” function adds statistical indicators on the basis of the “expected improvement” function to avoid the local optimal solution caused by excessive collection in the optimization process. Therefore, compared with a single “expected improvement” function, the Elps+ function can coordinate the degree of improvement with the computation time of the objective function, and determine the global optimal solution more effectively.
Step S3. After verifying the performance and stability of the reconstructed model, output the predicted results of aerosol chemical components.

### Embodiment 2

Based on the same invention concept, the invention also provides a reconstruction system of aerosol chemical components based on CNN-BILSTM-BO, which accurately reconstructs a variety of aerosol chemical components (ammonium salt NH4+, sulfate SO42—, nitrate NO3, organic matter OM and elemental matter EM) without relying on traditional chemical analysis techniques. Its purpose is to solve the problems of high cost of chemical component observation, missing data, and inconsistency of variable types in multi-source observation data. The model is developed based on complex deep neural networks, the core sets of which are convolutional neural network (CNN), bidirectional long short-term memory neural network (BiLSTM) and Bayesian Optimization (BO). Among them, CNN is used to mine the features between multiple variables, BiLSTM is used to capture the time characteristics of the model, and BO is responsible for optimizing the hyperparameters of the model to obtain the optimal reconstruction model of key chemical components of PM2.5 (CNN-BILSTM-BO). CNN-BILSTM-BO can characterize the complex nonlinear relationship between multi-source observation variables and target chemical components, and accurately describe the mass concentration information of target chemical components. In addition, the invention integrates the feature analysis function and can extract and screen the feature of the data. The invention modularizes the model so that the model has good flexibility and applicability and can be further developed and improved, including,

The data preprocessing module, initialization system, collecting multi-source environment observation data, preprocessing and extracting key feature variables, and defining the decision space of control parameters and hyperparameter optimization.

In the feature analysis and evaluation module, the pre-processed multi-source environment observation data is input into the CNN-BILSTM model for feature analysis. In this module, the convolutional neural network extracts spatial features, and uses the bidirectional long and short term memory neural network to process time series information and evaluate the performance of the model. In the feature analysis and evaluation module, the multi-variable time series of multi-source environment observation data is converted into a multi-variable array through data folding, so that each time step is carried out independent convolution operation, the convolution layer extracts local features along the horizontal and vertical directions, and ensures the stability of the output through batch normalization processing. The modified linear unit performs nonlinear processing on the data after batch normalization, the average pooling layer divides the data into multiple regions, the sequence expansion layer restores the time series structure, the flattening layer converts the spatial dimension into the variable dimension, and the BILSTM layer processes the time series data in a forward and backward way to capture the past and future information. Finally, the discard layer randomly removes a certain proportion of neurons, and the fully connected layer converts the output results into dimensions. And then the regression output layer outputs the predicted results of aerosol chemical components.

In the training optimization module, the hyperparameters of the CNN-BILSTM model are iteratively adjusted by Bayesian optimization algorithm, the model is trained, the optimal reconstructed model is generated and the prediction results of aerosol chemical components are output. In the training optimization module, a set of initial hyperparameters are randomly selected as the decision vector, and the prior probability distribution of the objective function is adopted. The initial value of the hyperparameter is calculated, the likelihood distribution of the Gaussian process regression model is updated using the actual observed values, and the posterior probability distribution of the objective function is derived. Based on the posterior probability distribution, the value of the acquisition function is maximized, the optimal sampling point is determined, and the prior probability distribution of the objective function is updated through continuous iteration. The optimal hyperparameter combination corresponding to the minimum value of the final objective function is the optimal solution.

As shown in FIG. 2, the operation flow of CNN-BILSTM-BO is executed by a series of modules. Among them:

Data Preprocessing Modules (SetModel, InitModel, InputData, and PrepareData) are responsible for preprocessing operations, including defining the model's control parameters and decision space for hyperparameter optimization, as well as cleaning data and preparing input data. It is worth noting that PrepareData randomly selects multi-source observation data for training, validation, and testing of the model, but it still maintains that the data is arranged in chronological order.

The Fanalysis and EvaluateModel modules are responsible for feature analysis (calculating the importance of feature) and model evaluation, respectively.

Finally, Training Optimization Modules (TrainingModel, BayesianOpt, and UpdateGPR) train and optimize the model iteratively until convergence conditions are reached (set to a fixed value of 50 iterations).

Although the invention has been disclosed as a better embodiment, it is not used to define the invention, and any person skilled in the field may make possible changes and modifications to the technical scheme of the invention by using the method and technical content disclosed above, within the spirit and scope of the invention. Therefore, any content not separated from the technical scheme of the invention, including any simple modification, equivalent change and modification of the above embodiments according to the technical essence of the invention belong to the protection scope of the technical scheme of the invention.

1. A reconstruction method of aerosol chemical components based on CNN-BILSTM-BO, comprising the following steps:
Step S1, collecting multi-source environmental observation data through observation equipment, and conduct pre-processing and extract key feature variables;
Step S2, inputting the pre-processed multi-source environment observation data into the CNN-BILSTM model for feature analysis, wherein, convolutional neural network extracts spatial features and uses bidirectional long short-term memory neural network to process time series information, and adjusts hyperparameters of the CNN-BILSTM through Bayesian optimization algorithm, a reconstructed model of aerosol chemical components is generated;
Step S3, after verifying the performance and stability of the reconstructed model, outputting the predicted results of the chemical components of the aerosol.

2. The reconstruction method of aerosol chemical components described in claim 1, wherein in step S2, the convolutional neural network extracts spatial features, including folding the multivariable time series of the multi-source environmental observation data into a multivariable array to complete independent convolution operations at each time step, Each time step of the multi-source environment observation data is independently convolved along the horizontal and vertical directions, local features are extracted by convolution kernel and normalized and nonlinear processing is performed by batch normalization and correction of linear elements, the time series structure of the multi-source environment observation data is reconstructed using the sequence expansion layer, and the spatial dimension is folded into the variable dimension by the flattening layer.

3. The reconstruction method of aerosol chemical components described in claim 1, wherein in step S2, the bidirectional long short-term memory neural network is used to process time series information, including that the bidirectional long short-term memory layer inputs the time characteristics of the data from the convolutional neural network in a forward and backward way, and simultaneously captures the past and future information, randomly, remove a certain percentage of neurons by dropping layers, the output result of the bidirectional long short term memory layer is dimensionally transformed to match the target output size using the fully connected layer, and finally the regression output layer outputs the regression estimate of the aerosol chemical components.

4. The reconstruction method of aerosol chemical components described in claim 3, wherein in step S2, the hyperparameters are adjusted by the Bayesian optimization algorithm, including,
the objective function of the CNN-BiLSTM model is constructed, and a set of initial hyperparameters are randomly selected in the decision space as the initial decision vector, the value of the objective function is calculated and the prior probability distribution is obtained, and the maximum number of iterations and calculation time are controlled by the control parameters,
the likelihood distribution of the Gaussian process regression model of the objective function is updated by the actual observed values, and the posterior probability distribution is derived to reflect the update of the objective function,
based on the posterior probability distribution, the acquisition function value is maximized to determine the next optimal sampling point, and the corresponding prior probability distribution is continued to be updated, iterating continuously until the combination of hyperparameters corresponding to the minimum value of the objective function is determined, and the hyperparameter combination is the corresponding optimal solution.

5. The reconstruction method of aerosol chemical components mentioned in claim 4, wherein in step S211, the objective function of the CNN-BILSTM model is constructed, and a set of initial hyperparameters are randomly selected in the decision space as the initial decision vector, the value of the objective function is calculated and the prior probability distribution is obtained, including,
$$\begin{matrix}p ( f ( x \vert input ) \vert Y ) = p ( Y \vert f ( x \vert input ) ) p ( f ( x \vert input ) ) / p ( Y ) : \\ {x}^{*} = arg min f ( x \vert input ) , x ∈ X ⊆ {R}^{d} :\end{matrix}$$
wherein, x is the decision vector composed of d hyperparameters, X is the decision space, input is the input data, x* is the optimal hyperparameter combination vector, f(x|input) is the objective function, Y is the actual observation value, p(Y|f(x|input)) is the likelihood distribution, p(f(x|input)) is the prior probability distribution, that is, the estimate of f(x|input), p(Y) is the edge likelihood distribution, p(f(x|input)|Y) is the posterior probability distribution, that is, the confidence coefficient of f(x|input).

6. The reconstruction method of aerosol chemical components described in claim 5, wherein in step S211, the likelihood distribution of the Gaussian process regression model of the objective function is updated by actual observed values, and a posterior probability distribution is derived from it to reflect the update of the objective function, including,
$$\begin{matrix}f ( x \vert input ) \sim GP ( mean ( x ) , k ( x , {x}^{′} ; θ ) ) ; \\ k ( x , {x}^{′} ; θ ) = {θ}_{1} ( 1 + \frac{\sqrt{5} \vert x - {x}^{′} \vert}{{θ}_{2}} + \frac{5 {( x - {x}^{′} )}^{2}}{3 {θ}_{2}} ) exp ( - \frac{\sqrt{5} \vert x - {x}^{′} \vert}{{θ}_{2}} ) ; \\ {θ}_{1} = {σ}_{f}^{2} , {σ}_{f} > 0 ; \\ {θ}_{2} = {σ}_{1} , {σ}_{1} > 0 ;\end{matrix}$$
wherein, GP(mean(x),k(x,x′;θ)) is the Gaussian process regression model, which is composed of mean value function(mean(x)) and covariance kernel function(k(x,x′;θ)), k(x,x′;θ) is The Matern 5/2 covariance kernel function, where x and x′ is expressed as any two coordinate points, θ is the kernel parameter vector, σ_{f} is the signal standard deviation, and σ₁ is the feature length scale.

7. The reconstruction method of aerosol chemical components described in claim 6, wherein based on the posterior probability distribution, the acquisition function value is maximized to determine the next optimal sampling point, and the corresponding prior probability distribution is continuously updated, and the continuous iteration is continued until the hyperparameter combination corresponding to the minimum value of the objective function is determined, the hyperparameter combination is the corresponding optimal solution, including,
$$\begin{matrix}EI ( x , P ) = {E}_{P} [ max ( 0 , {μ}_{P} ( {x}_{best} ) - f ( x \vert input ) ) ] ; \\ EIpS ( x ) = {EI}_{P} ( x ) / {μ}_{s} ( x ) ; \\ {σ}_{P}^{2} ( x ) = {σ}_{F}^{2} ( x ) + {σ}^{2} , {σ}_{F} ( x ) < {t}_{σ} σ ;\end{matrix}$$
where, El(x,P) is the acquisition function, x is the same as x in the objective function(f(x|input)), P is the objective function(p(f(x|input))), μ_{P}(x_{best}) is the minimum posterior mean, x_{best} is the currently known optimal solution, ElpS(x) represents the expected improvement function per second and μₛ(x) is the posterior mean of the time Gaussian process model,
$${σ}_{P}^{2} ( x )$$
represents the audition function, σ_{F}(x) is the standard deviation of f(x|input), σ is the posteriori standard deviation of the additional noise, and t_{σ} is the value of the mining proportion in the acquisition function.

8. A reconstruction system of aerosol chemical components based on CNN-BILSTM-BO adopts the reconstruction method of aerosol chemical components described in claim 1-7, comprising,
the data preprocessing module initializes the system, collects multi-source environment observation data, preprocesses and extracts key feature variables, and defines the decision space of control parameters and hyperparameter optimization,
in the feature analysis and evaluation module, the pre-processed multi-source environment observation data is input into the CNN-BILSTM model for feature analysis, in this module, the convolutional neural network extracts spatial features, uses the bidirectional long short-term memory neural network to process time series information, and evaluates the performance of the model,
the model training optimization module iteratively adjusts the hyperparameters of the CNN-BILSTM model by Bayesian optimization algorithm, to train the model and then generates the optimal reconstructed model and outputs the prediction results of aerosol chemical components.

9. The reconstruction system of aerosol chemical components mentioned in claim 8, wherein in the characteristic analysis and evaluation module, the multi-variable time series of the multi-source environmental observation data is converted into a multi-variable array through data folding, so that each time step is independently convolution operation, and the convolution layer extracts local features along the horizontal and vertical directions, in addition, the output stability is ensured by batch normalization, the modified linear unit performs nonlinear processing on the batch normalized data, the average pooling layer divides the data into multiple regions, the sequence expansion layer restores the time series structure, the flattening layer converts the spatial dimension into variable dimension, and the BiLSTM layer processes the time series data in both forward and backward ways, after capturing past and future information, the discard layer randomly removes a certain proportion of neurons, and the fully connected layer converts the output results into dimensions, finally, the regression output layer outputs the predicted results of aerosol chemical components.

10. The reconstruction system of aerosol chemical components mentioned in claim 9, randomly selecting a set of initial hyperparameters as the decision vector in the model training optimization module, calculating the initial value of the hyperparameters through the prior probability distribution of the objective function, and updating the likelihood distribution of the Gaussian process regression model with actual observed values, the posterior probability distribution of the objective function is derived, and based on the posterior probability distribution, the value of the acquisition function is maximized, the optimal sampling point is determined, and the prior probability distribution of the objective function is updated continuously, and the optimal hyperparameter combination corresponding to the minimum value of the objective function is finally the optimal solution.
